(19) United States
(12) Patent Application Publication
(10) Pub. No.: US 2025/0156932 A1
(43) Pub. Date: May 15, 2025

(54) SYSTEMS AND METHODS FOR OUTPUTTING RECOMMENDED FOOD ITEMS

(71) Applicant: EBudget Ltd, Oxford (GB)

(72) Inventor: Ellie Doroudian, Oxford (GB)

(21) Appl. No.: 18/949,207

(22) Filed: Nov. 15, 2024

(30) Foreign Application Priority Data
Nov. 15, 2023 (GB) 2317486.5

(51) Int. Cl. G06Q 30/0601; G01C 21/34; G06Q 10/087; G16H 20/60

(52) U.S. Cl. CPC G06Q 30/0639; G01C 21/3484; G06Q 10/087; G06Q 30/0631; G16H 20/60

(57) ABSTRACT

There is provided a computer-implemented method for determining a navigation route configured to obtain a plurality of products sought by a user. The method comprises obtaining an indication of a geographical location of a user; obtaining an indication of a plurality of products sought by the user; and obtaining a plurality of indications based on the indication of the plurality of products sought by the user, wherein each of the plurality of indications relates to an available product, and wherein each indication of an available product comprises an indication of the geographical location of said available product. The method then comprises grouping the plurality of indications each relating to an available product into a plurality of destinations based on the geographical location of the available products, and determining a plurality of navigation routes from the geographical location of the user, wherein each navigation route is configured to navigate to at least one destination of the plurality of destinations, such that each complete navigation route is configured to navigate to a plurality of available products corresponding to each of the plurality of products sought by the user. A weighting is applied to each of the plurality of navigation routes, wherein the weighting is inversely proportional to at least one of (i) time and (ii) distance of the navigation route; and at least one recommended navigation route is output, wherein the navigation route is configured to obtain the plurality of products sought by the user, based on the first weighting applied to each of the plurality of navigation routes.

### FIELD OF THE INVENTION

The present disclosure relates to systems and methods for outputting recommended food items to improve access to food items, for example based on location, food quality, and nutritional requirements.

### BACKGROUND

As the global population rises, there is increasing pressure on food supply chains to provide for and feed the world. Despite this, it is reported that UK supermarkets throw away approximately 100,000 tonnes of edible food each year-equivalent to 250 million meals going uneaten. Instead, this food ends up in animal feed, used for anaerobic digestion, or even going to landfill, with devastating environmental consequences.

It is known that diet contributes to the development of many chronic health conditions, including but not limited to obesity, cardiovascular disease, hypertension, stroke, type 2 diabetes, metabolic syndrome, some cancers, and even some neurological diseases. In particular, it is well established that nutrient deficiencies or suboptimal nutritional status can contribute to the development of diseases and/or health problems. While dietary modifications can help to prevent the development of many chronic diseases, once a condition has developed, changes to a person's usual diet are often needed to assist with disease management.

Despite the understood importance of diet on our health, it is reported that more than half of the energy (calories) consumed by an average person in the UK comes from ultra-processed foods.

There is therefore a need to provide consumers with improved access to nutritionally rich foods to improve their health, whilst also reducing food waste by supermarkets and food suppliers.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

In a first aspect of the invention, there is provided a computer implemented method for determining a navigation route configured to obtain a plurality of products sought by a user. The method comprises obtaining an indication of a geographical location of a user, and obtaining an indication of a plurality of products sought by the user. The method then comprises retrieving a plurality of indications based on the indication of the plurality of products sought by the user, wherein each of the plurality of indications relates to an available product, and wherein each indication of an available product comprises an indication of the geographical location of said available product. The method then groups the plurality of indications each relating to an available product into a plurality of destinations based on the geographical location of the available products, for example such that indications relating to available products at the same geographical location are grouped together.

The method then further comprises determining a plurality of navigation routes from the geographical location of the user, wherein each navigation route is configured to navigate to at least one destination of the plurality of destinations, such that each complete navigation route is configured to navigate to a plurality of available products corresponding to each of the plurality of products sought by the user. For example, if a first destination, shop A, comprises a plurality of indications relating to available products, wherein the plurality of indications correspond to each of the plurality of products sought by a user, a navigation route may be configured to navigate to the first destination only. However, by contrast, if a first destination, shop A, comprises a plurality of indications relating to available products, wherein the plurality of indications correspond to a portion of the plurality of products sought by a user, and a second destination, shop B, comprises at least one indication relating to at least one available product, wherein the at least one indication corresponds to the portion of the plurality of products sought by a user not present at the first destination, shop A, a navigation route may be configured to navigate to the first destination, shop A, and the second destination, shop B.

A first weighting is applied to each of the plurality of navigation routes, wherein the first weighting is inversely proportional to at least one of (i) time and (ii) distance of the navigation route.

The method may then output at least one recommended navigation route, and/or at least one recommended destination, configured to obtain the plurality of products sought by the user, based on the first weighting applied to each of the plurality of navigation routes.

Applying a first weighting which is inversely proportional to at least one of (i) time and (ii) distance of the navigation route may be advantageous to favourably weight navigation routes which are geographically close to the user, or quick for the user to navigate to. This may be advantageous to reduce carbon emissions arising from travel. For example, the method may favourably weight a first navigation route that navigates to a first destination, shop A, to obtain a first portion of products sought by the user, and a second destination, shop B, to obtain the remaining second portion of products sought by the user, the first navigation route being 0.5 miles, compared to a second navigation route that navigates to a third destination, shop C, to obtain all products sought by the user, wherein the second navigation route is 2 miles.

In some examples, at least a portion of the plurality of navigation routes are configured to navigate to two or more destinations, such that each complete navigation route is configured to navigate to a plurality of available products corresponding to each of the plurality of products sought by the user. This may be advantageous to navigate to each of the plurality of products sought by the user in instances where the plurality of products are available from different destinations.

Determining the plurality of navigation routes may further comprise determining a plurality of navigation routes from the geographical location of the user for a plurality of modes of transport. The method may then further comprise applying a second weighting to each of the plurality of navigation routes, wherein the second weighting is based on the mode of transport of the route; and outputting the at least one recommended navigation route, and/or at least one recommended destination, based on the first weighting and the second weighting. Applying the second weighting based on the mode of transport of the route may be advantageous to favourably weight navigation routes reliant on favourable modes of transport. This may be advantageous to reduce carbon emissions arising from travel, for example by favourably weighting navigation routes for low-carbon modes of transport, such as walking or cycling, compared to navigation routes for higher-carbon modes of transport, such as private car transportation. For example, the method may favourably weight a first navigation route that navigates to a first destination, shop A, to obtain the plurality of products sought by the user, the first navigation route being a 15-minute walk, compared to a second navigation route that navigates to a second destination, shop B, to obtain the products sought by the user, wherein the second navigation route is 5-minute drive.

Each indication of the plurality of indications relating to an available product may relate to one of: (i) an available product corresponding to one of the plurality of products sought by the user; and (ii) an available substitute product corresponding to one of the plurality of products sought by the user. Purely as an example, if one of the products sought by the user is sourdough bread, an indication relating to an available product corresponding to one of the plurality of products sought by the user comprises an indication relating to sourdough bread, whereas an indication relating to an available substitute product corresponding to one of the plurality of products sought by the user may comprise an indication relating to, for example, soda bread, rye bread, granary bread, or white bread, etc.

In some examples, an indication relating to an available substitute product corresponding to one of the plurality of products sought by the user may be obtained based on having similar ingredients to at least one of the products sought by the user. In particular, an indication relating to an available substitute product corresponding to one of the plurality of products sought by the user may be selected from a plurality of indications each relating to an available product based on a determination that the indication relating to an available substitute product has the highest number of common ingredients with one of the plurality of products sought by the user, in particular wherein said one of the plurality of products sought by the user is unavailable. For example, if at least one of the products sought by the user, such as sourdough bread, is not available at a destination, the method may identify a plurality of available products of the same product category as the missing product at said destination, such as bread products available at said destination. The method may then compare the ingredients of the available bread products to the ingredients of the missing product sought by the user. A recommended available substitute product can then be identified based on the available bread product having the most similar ingredients to the missing product sought by the user. For example, if the available bread products at said destination comprised rye bread, soda bread, and granary bread, and the missing product sought by the user was sourdough bread, the recommended available substitute product may be identified as the available soda bread due to the most similar ingredients. This recommended available substitute product may then be included in a navigation route to obtain the products sought by the user, as described herein.

The method may then further comprise, for each of the plurality of navigation routes, determining the number of available substitute products for the navigation route, and applying a third weighting to each of the plurality of navigation routes, wherein the third weighting is inversely proportional to the number of available substitute products for the navigation route. Outputting the at least one recommended navigation route, and/or at least one recommended destination, may then additionally be based on the third weighting applied to each of the plurality of navigation routes. This may be advantageous to favourably weight navigation routes and/or destinations that navigate to fewer substitute products. However, this is combined with the first weighting, and optionally the second weighting, the ensure that outputted recommended navigation route, and/or at least one recommended destination, also considers the (i) time and (ii) distance of the navigation route, and optionally the mode of transport of the navigation route. As such, the method may still recommend a first navigation route that navigates to a first destination, shop A, to obtain a first portion of available products sought by the user, and a second destination, shop B, to obtain the remaining second portion of available substitute products sought by the user, the first navigation route being 0.5 miles, compared to a second navigation route that navigates to a first destination, shop A, to obtain a first portion of available products sought by the user, and a second destination, shop C, to obtain the remaining second portion of available products sought by the user, wherein the second navigation route is 2 miles. However, the method may recommend a third navigation route relative to the first navigation route, wherein the third navigation route is configured to navigate to a first destination, shop A, to obtain a first portion of available products sought by the user, and a second destination, shop D, to obtain the remaining second portion of available products sought by the user (i.e. having fewer substitute products relative to the first navigation route), wherein the third navigation route is 0.7 miles.

Alternatively, the method may comprise identifying at least one destination which does not 5 comprise an indication relating to an available product for all of the plurality of products sought by the user; and, for said destination, determining at least one missing product of the plurality of products sought by the user, wherein said destination does not comprise an indication relating to an available product for the at least one missing product. The method may then comprise retrieving, for said destination, a second plurality of indications each relating to an available substitute product, wherein the second plurality of indications are retrieved based on the at least one missing product. A second plurality of navigation routes from the geographical location of the user may then be determined, wherein each navigation route of the second plurality of navigation routes is configured to navigate to at least one destination of the plurality of destinations, such that each complete navigation route is configured to navigate to (i) at least one available product, and (ii) at least one available substitute product, such that the at least one available product and the at least one available substitute product correspond to each of the plurality of products sought by the user. The first weighting is also applied to each of the second plurality of navigation routes, wherein the weighting is inversely proportional to at least one of (i) time and (ii) distance of the navigation route. A third weighting is also applied to each of the second plurality of navigation routes, wherein the third weighting is inversely proportional to the number of available substitute products for the navigation route. Outputting the at least one recommended navigation route, and/or at least one recommended destination, is based on the weightings applied to each of the plurality of navigation routes, including the first and third weightings applied to the second plurality of navigation routes.

Optionally, the method may further comprise applying the second weighting, discussed above, to the second plurality of navigation routes, wherein the second weighting is based on the mode of transport of the route.

Each indication of an available product may further comprise an indication of at least one of property of said product. The method may further comprise obtaining a set of user preferences, wherein the set of user preferences correspond to preferred properties of at least a portion of the plurality of products sought by the user. For example, the set of user preferences may include, but are not limited to, indications of preferred brands of product, and/or preferred varieties or flavours of products, etc.

The method may further determine a first weighting for each indication of an available product, based on the at least one property of the available product and the set of user preferences. This may be advantageous to favourably weight indications of available products that match the set of user preferences.

For each of the plurality of navigation routes, the method may then determine a fourth weighting, wherein the fourth weighting is based on the first weighting of each indication of an available product navigated to by the complete navigation route. Outputting the at least one recommended navigation route, and/or at least one recommended destination, may then additionally be based on the fourth weighting applied to each of the plurality of navigation routes. This may be advantageous to favourably weight navigation routes, and/or destinations therein, that comprises available products corresponding to the set of user preferences.

The method may then further comprise, for each of the plurality of navigation routes, applying a fifth weighting to each of the plurality of navigation routes, wherein the fifth weighting is inversely proportional to the number of destinations within the complete navigation route. This may be advantageous to favourably weight navigation routes having fewer different destinations that a user has to visit, for example the method may be configured to favour a route that requires only two destinations, compared to a navigation route that requires the user to visit four different destinations in order to obtain the plurality of desired products.

Each indication of the plurality of indications relating to an available product may further comprise an indication of the expiry date or ‘best before’ date of the product, such as a food item. The method may then further comprise, for each of the plurality of navigation routes, applying a sixth weighting to each of the plurality of navigation routes, wherein the sixth weighting is based on the expiry date of each available product within the complete navigation route. For example, navigation routes that navigate to a plurality of available products having expiry dates on or closer to the current date may be more favourably weighted than navigation routes that navigate to a plurality of available products which have a longer shelf life and/or distant expiry date. This may be advantageous to favourably weight navigation routes comprising products which are soon to expire as this promotes the sale and use of items which are soon to expire, and therefore reduces potential food waste.

Whilst the above discloses first, second, third, fourth weightings, and so on, the skilled person will understand that this nomenclature is merely intended to differentiate the plurality of weightings which may be used with the present invention. As such, the skilled person will understand that the weightings need not be applied in any particular order, and any combination of weightings may be applied. Purely for illustration, an embodiment of the present invention may apply the first weighting and the sixth weighting, for example, not the second to fifth weightings described above.

In another aspect of the invention, there is provided a computer-implemented method for outputting an indication of at least one recommended available product from a plurality of available products, wherein the plurality of available products are available at different locations. The method comprises obtaining an indication of a geographical location of a user, and obtaining an indication of a product sought by a user. The method then retrieves a plurality of indications each relating to an available product, wherein the plurality of indications are retrieved based on the indication of the product sought by the user. Each indication of an available product comprises an indication of the product price, and an indication of the geographical location of the product.

The method applies a first weighting to each of the plurality of indications relating to an available product, based on the indication of the product price.

For each of the plurality of indications relating to an available product, the method obtains at least one navigation route from the geographical location of the user to the geographical location of the available product. In some examples, obtaining the at least one navigation route may comprise determining at least one navigation route from the geographical location of the user to the geographical location of the available product.

The method then applies a second weighting to each navigation route, wherein the second weighting is inversely proportional to at least one of (i) time and (ii) distance of the navigation route to the geographical location of the available product. An indication of at least one recommended available product is then output, based on the first weighting and the second weighting.

This may be advantageous to recommend an available product from a plurality of available products, wherein the plurality of available products are available at different locations, based on both cost and at least one of (i) time and (ii) distance to the geographical location of the available product. This may be advantageous to reduce unnecessary travel time to obtain a desired product, and reduce carbon emissions resulting from travel.

In some examples, obtaining or determining the at least one navigation route from the geographical location of the user to the geographical location of the product comprises obtaining or determining a plurality of navigation routes for a plurality of modes of transport. The second weighting applied to each navigation route may then additionally be based on the mode of transport of the navigation route. For example, the second weighting applied to each navigation route may comprise: a first component being inversely proportional to at least one of (i) time and (ii) distance of the navigation route to the geographical location of the available product, and a second component based on the mode of transport of the route. This may be advantageous to reduce carbon emissions arising from travel to obtain a desired product, for example the second component of the second weighting may be configured to favourably weight navigation routes for low-carbon modes of transport, such as walking or cycling, compared to navigation routes for higher-carbon modes of transport, such as private car transportation. For example, the method may favourably weight a first navigation route that navigates to a first destination, shop A, to obtain a product sought by the user, the first navigation route being a 15-minute walk, compared to a second navigation route that navigates to a second destination, shop B, to obtain the product sought by the user, wherein the second navigation route is 5-minute drive.

In some examples, the method may further comprise, based on the mode of transport of the route, calculating a cost of travel to obtain the product for each navigation route, wherein the cost of travel is based on the at least one navigation route and the mode of transport. The second weighting may then additionally comprise weighting component which is inversely proportional to the cost of travel. The cost of travel may be based on the mode of transport, for example, the cost of travel for a private car may be determined based on the distance of travel and fuel price, whereas the cost of travel via public transport may be based on the public transport fare, such as a bus or train. The cost of travel may incorporate the cost of travel from the user's geographical location to the product geographical location, and optionally, the cost of travel from the product geographical location to an end destination, such as the user's home address.

In another aspect of the invention, there is provided a computer-implemented method for outputting at least one recommended food item to reduce nutritional deficiencies. The method comprises obtaining an indication of a dietary requirement of a user, and obtaining a set of threshold nutritional values corresponding to nutritional indicators, wherein the set of threshold nutritional values is based on the indication of the dietary requirement of the user. The method then comprises obtaining an indication of a plurality of food items that the user intends to purchase, and retrieving a set of nutritional indicators for each of the plurality of food items that the user intends to purchase. The method determines a set of aggregate nutritional values corresponding to the set of nutritional indicators for each of the plurality of food items that the user intends to purchase, and compares the set of aggregate nutritional values to the set of threshold nutritional values. In the event that at least one of the aggregate nutritional values from the set of aggregate nutritional values is different to the corresponding threshold nutritional value from the set of threshold nutritional values, the method outputs at least one recommended food item, wherein the at least one recommended food item is identified and output based on a set of nutritional indicators for the at least one recommended food item, and the difference between the set of aggregate nutritional values and the set of threshold nutritional values.

This may be advantageous to output recommended food items to ensure a user's recommended nutritional requirements are being met based on their dietary requirements, thereby helping to reduce nutritional deficiencies of the user.

Optionally, the outputted at least one recommended food item may be a substitute food item corresponding to at least one of the plurality of food items that the user intends to purchase.

The method may output the at least one recommended food item in the event that at least one of the aggregate nutritional values from the set of aggregate nutritional values is less than the corresponding threshold nutritional value from the set of threshold nutritional values.

Alternatively, the method may output the at least one recommended food item in the event that at least one of the aggregate nutritional values from the set of aggregate nutritional values is greater than the corresponding threshold nutritional value from the set of threshold nutritional values. Preferably, in the event that at least one of the aggregate nutritional values from the set of aggregate nutritional values is greater than the corresponding threshold nutritional value from the set of threshold nutritional values, the method may output at least one recommended substitute food item corresponding to at least one of the plurality of food items that the user intends to purchase, based on a set of nutritional indicators for the at least one recommended substitute food item, and the difference between the set of aggregate nutritional values and the set of threshold nutritional values. This may be advantageous to promote food items better suited to the nutritional requirements of a user.

In some examples, the method may comprise substituting at least one of the plurality of food items that the user intends to purchase with the at least one recommended substitute food item in an order of food items executed by the user.

In another aspect of the invention, there is provided a computer-implemented method for outputting at least one recommended food item to reduce nutritional deficiencies. The method comprises obtaining an indication of a dietary requirement of a user, and obtaining a set of weightings based on the indication of the dietary requirement of the user, wherein the set of weightings corresponds to a set of nutritional indicators. For each of a plurality of food items, the method retrieves a set of nutritional indicators for the food item, and scores each of the plurality of food items based on (i) the set of nutritional indicators for the food item, and (ii) the set of weightings based on the indication of the dietary requirement of the user. The method then outputs at least one recommended food item, based on the determined score. In some examples, a set of ingredients for each of the plurality of food items is also retrieved. For each of a plurality of food items, the method may then compare the set of ingredients for the food item to a predetermined set of ingredients to identify a number of ingredients common to the set of ingredients for the food item and the predetermined set of ingredients. Scoring each food item may then additionally be based on (iii) the number of ingredients common to the set of ingredients for the food item and the predetermined set of ingredients. For example, scoring each food item may then additionally be based on a scoring component, wherein the scoring component is inversely proportional to the number of ingredients common to the set of ingredients for the food item and the predetermined set of ingredients, for example wherein the predetermined set of ingredients may comprise ingredients associated with health risks, such as E numbers, artificial sweeteners, etc., or allergens.

In another aspect of the invention, there is provided a computer-implemented method for outputting at least one recommended food item to reduce nutritional deficiencies. The method comprises obtaining an indication of a food item that the user intends to purchase; and retrieving a plurality of indications each relating to an available food item corresponding to the food item that the user intends to purchase, wherein each of the plurality of indications comprises a set of ingredients for the available food item and an indication of the product price. For each of the plurality of indications, the method then compares the set of ingredients for the available food item to a predetermined set of ingredients to identify a number of ingredients common to the set of ingredients for the food item and the predetermined set of ingredients. A first weighting is applied to each of the plurality of indications based on the number of ingredients common to the set of ingredients for the food item and the predetermined set of ingredients. A second weighting is also applied to each of the plurality of indications, based on the indication of the product price. The method then outputs at least one recommended food item from the plurality of indications each relating to an available food item, based on the first weighting and second weighting.

The skilled person will understand from the discussion above that a “first weighting” and a “second weighting” etc., may refer to a “first set of weightings” and a “second set of weightings” etc., wherein each of the weightings applied to each of the plurality of indications (or navigation routes, etc.) are different based on the characteristics of that indication (or navigation route, etc.). It is not required for a “first weighting”, “second weighting”, and so on, to be a fixed value.

A computer readable non-transitory storage medium is also provided herein, the computer readable non-transitory storage medium comprising a program for a computer configured to cause a processor to perform the method of any preceding aspect of the invention.

In another aspect of the invention, there is also provided an inventory monitoring device, comprising a sensor configured to be mounted to a shelf, wherein the sensor is configured to obtain an indication of the weight of product stock on the shelf, and a processor configured to detect when at least one product is removed from the shelf based on an indication of the weight of product stock on the shelf obtained by the sensor. The inventory monitoring device further comprises a communication means configured to send a signal to a remote device, such as a remote server, wherein the signal includes the indication of the at least one product which has been removed from the shelf in the store, for example wherein the communication means may be a wireless communication means.

The signal sent to the remote device may be configured to update a record of inventory, for example by deducting at least one product from the record of inventory based on the signal.

This inventory monitoring device may be advantageous compared to alternative solutions which rely on monitoring via camera-based solutions as the weight-based sensor does not require continual image capture which can cause data handling burdens for stores, particularly where the camera-based solutions may inadvertently capture faces or identifying features of members of the general public. Camera-based solutions are also often prohibitively complex and expensive to install.

The sensor may be configured to be installed on a shelf, such that the product stock is stored on top of the sensor, for example wherein the sensor is configured to be arranged between the product stock and the shelf itself in use. In such embodiments, the sensor may be configured to obtain an indication of the weight of product stock directly.

Alternatively, the sensor may be configured to be installed into a supporting structure, such as a bracket, wherein the supporting structure is configured to support the shelf. In such embodiments, the sensor may be configured to obtain an indication of the weight of product stock indirectly, based on obtaining an indication of the weight of the shelf and the product stock stored on the shelf.

The processor may be configured to detect when at least one product is removed from the shelf based on a change in the indication of the weight of product stock on the shelf. Preferably, the processor is configured to detect a number of products that have been removed from the shelf based on a change in the indication of the weight of product stock on the shelf, and an indication of the weight of each product from the product stock.

The signal may further comprise an indication of the number of products that have been removed from the shelf, wherein sending the signal to the remote device may be configured to update a record of inventory by deducting a number of products from the record of the inventory, based on the indication in the signal of the number of products that have been removed.

In some examples, the record of inventory be updated based on both the signal from the inventory monitoring device, and a signal from a till or cash register, wherein the till or cash register includes an indication that the at least one product which has been removed from the shelf in the store has been purchased using the till or cash register. This may improve the accuracy of the record of inventory, as the record of inventory may only be updated based on receipt by the remote device, such as the remote server, of both signals from the inventory monitoring device, and the till or cash register.

In addition, the record of inventory may also be updated based on the signal from the inventory monitoring device alone, in particular in the event that no signal is received from the till or cash register within a predetermined time period from receiving the signal from the inventory monitoring device. For example, if a signal is sent to the remote device by the inventory monitoring device, but no signal is received from the till or cash register to indicate that the at least one product which has been removed from the shelf in the store has been purchased within an hour, the record of inventory may still be updated, and the item may be logged as stolen. This may be advantageous to help stores track and monitor occurrences of shop lifting and theft, and implement preventative measures.

In some examples, the inventory monitoring device may further comprise a camera configured to obtain images of product stock on the shelf, wherein the processor is further configured to detect an indication of the at least one product which has been removed from the shelf in the store based on at least a portion of the images obtained by the camera. The camera may be configured.

### DRAWINGS

Embodiments of the disclosure will now be described, by way of example only, with reference to the accompanying drawings, in which:

FIG. 1 shows a schematic flow diagram illustrating an example method of the present invention.

FIG. 2 shows a schematic of an example system configured to perform the method of the present invention, such as the method of FIG. 1.

FIG. 3 shows a box diagram of an example user equipment device for use in the system of FIG. 2.

FIG. 4 shows a schematic flow diagram illustrating another example method of the present invention.

### SPECIFIC DESCRIPTION

Embodiments of the claims relate to systems and methods for outputting recommended food items to improve access to food items, for example based on location, food quality, and nutritional requirements.

It will be appreciated from the discussion above that the embodiments shown in the Figures are merely exemplary, and include features which may be generalised, removed, or replaced as described herein and as set out in the claims.

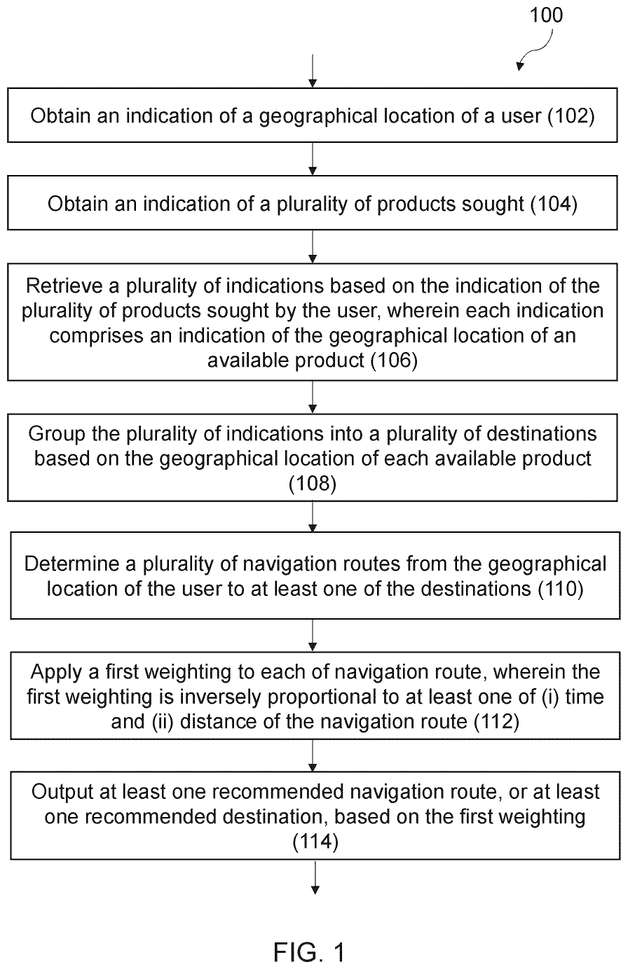

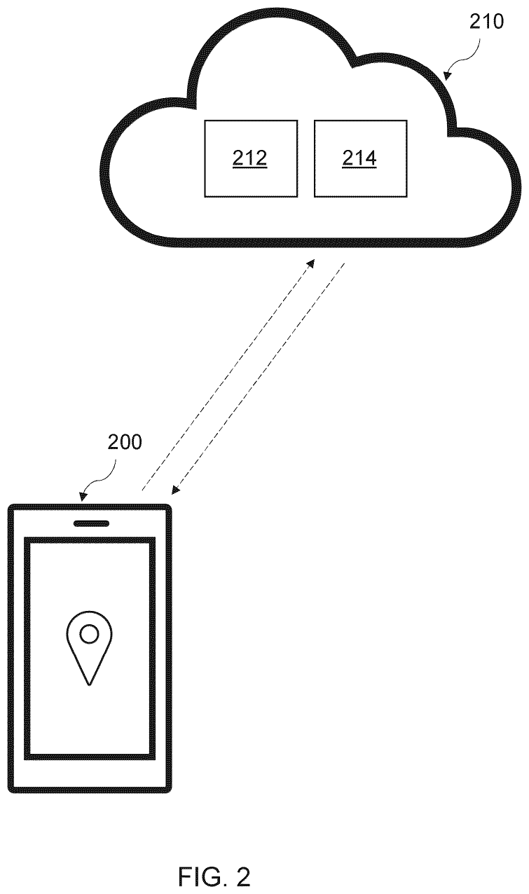

FIG. 1 shows a schematic flow diagram illustrating an example method 100 of the present invention. This is described with reference to FIG. 2 which shows a schematic of an example system configured to perform the method 100.

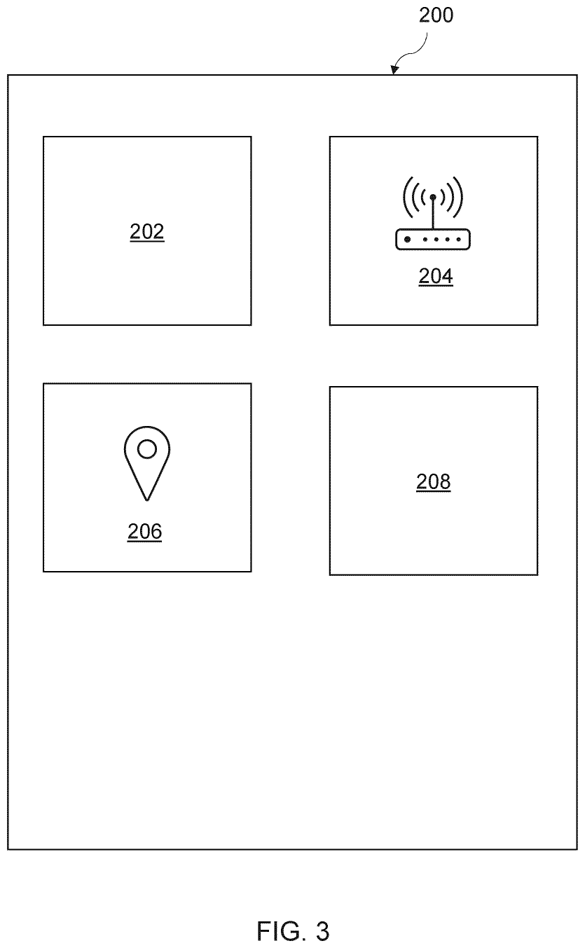

The system of FIG. 2 comprises a user equipment device 200, such as but not limited to a smartphone, and a remote server 210. As shown in FIG. 3, the user equipment device 200 comprises a processor 202, and a wireless communication means 204. The wireless communication means 204 is configured for wireless communication with the remote server 210. As an example, the wireless communication means 204 may be configured for wireless mobile telecommunications, such as but not limited to 3G, 4G, or 5G communications. The user equipment device 200 further comprises a means for determining geographical location 206, such as but not limited to a GPS receiver. The user equipment device 200 also comprises a local memory 208.

The remote server 210 also comprises a processor 212 and a memory 214.

In an embodiment of the present invention, the processor 212 obtains an indication of the geographical location of the user equipment device 200, and thereby the geographical location of the user (102). The indication of the geographical location is determined by the means for determining geographical location 206 at the user equipment device 200 and sent to the remote server 210 via the wireless communication means 204.

The processor 212 also obtains an indication of a plurality of products sought by the user (104). This may be obtained via user input into the user equipment device 200, for example via a touch screen, keypad, or any other user input means. This obtained user input may then be sent as a signal to the remote server 210 via the wireless communication means 204.

Based on the indication of a plurality of products sought by the user, the processor 212 is configured to retrieve a plurality of indications from the memory 214, wherein each of the plurality of indications relates to an available product (106). Each indication of an available product also comprises an indication of the geographical location of said available product, and an indication of the price of the available product. The price of the product may be an indication of the real-time price of the available product to reflect any current promotional offers at a given destination.

The processor 212 groups said plurality of indications into a plurality of destinations, based on the geographical location of each available product (108). Each destination may correspond, for example, to a store comprising at least one available product.

Purely for illustration, the indication of a plurality of products sought by the user may comprise a signal indicating the user seeks to buy (i) semi-skimmed milk, (ii) sourdough bread, and (iii) digestive biscuits.

The retrieved and grouped plurality of indications each correspond an available product based on the indication of products sought by the user. In particular, each indication of the plurality of indications relating to an available product may relate to one of: (i) an available product corresponding to one of the plurality of products sought by the user; and (ii) an available substitute product corresponding to one of the plurality of products sought by the user. For example:
(i) semi-skimmed milk, shop A, [symbol] 1.89;
(ii) digestive biscuits, shop A, [symbol] 1.20;
(iii) whole milk (substitute), shop B, [symbol] 1.89;
(iv) sourdough bread, shop B, [symbol] 3.99;
(v) digestive biscuits, shop B, [symbol] 1.00;
(vi) semi-skimmed milk, shop C, [symbol] 1.50;
(vii) sourdough bread, shop C, [symbol] 3.50;
(viii) digestive biscuits, shop C, [symbol] 1.15;

The processor 212 then determines a plurality of navigation routes, each navigation route is configured to navigate from the geographical location of the user to at least one of the destinations (110), such that each navigation route is configured to navigate to a plurality of available products corresponding to each of the plurality of products sought by the user. For example, following the example above, each navigation route is configured to navigate to one available product for each of (i) semi-skimmed milk, (ii) sourdough bread, and (iii) digestive biscuits.

Some example navigation routes have been included in table 1 below.

TABLE 1

| | Destination | | | | | |
|---|---|---|---|---|---|---|
| | to obtain | Destination | Destination | | | |
| | semi- | to obtain | to obtain | | | |
| Nav. | skimmed | sourdough | digestive | Nav. route | Distance | Mode of |
| route | milk | bread | biscuits | destinations | (miles) | transport |
| 1 | Shop A | Shop B | Shop A | Shop A; | 0.5 | By foot |
| | | | | Shop B | | |
| 2 | Shop A | Shop B | Shop B | Shop A; | 0.5 | By foot |
| | | | | Shop B | | |
| 3 | Shop B | Shop B | Shop B | Shop B | 0.4 | By foot |
| | (substitute) | | | | | |
| 4 | Shop C | Shop C | Shop C | Shop C | 3 | Bus |
| 5 | Shop C | Shop B | Shop B | Shop B; | 3.2 | Car |
| | | | | Shop C | | |
| 6 | Shop A | Shop C | Shop A | Shop A; | 3.5 | Car |
| | | | | Shop C | | |

The processor 212 then applies a plurality of weightings to each navigation route, and outputs at least one recommended navigation route, or plurality of destinations within a navigation route, based on the plurality of weightings.

In this example, a first weighting is applied wherein the first weighting is inversely proportional to the distance of the navigation route (112). A second weighting is also applied based on an indication of the mode of transport for the navigation route, wherein the second weighting is inversely proportional to the carbon emissions resultant from the mode of transport. A third weighting is also applied, wherein the second weighting is inversely proportional to an indication of cost of the mode of transport for the navigation route. An indication of cost of the mode of transport may be determined based on average cost per unit distance, or alternatively based on fare price for public transport. For example, to travel 3.2 miles in a car at an average cost of 16 pence per mile for a petrol car, an indication of the cost of travel for navigation route 5 may be determined to be approximately 51 pence. As another example, to travel 2 stops on a bus route to arrive at Shop C via navigation route 4, the indication of the cost of travel may be determined to be approximately [symbol] 1.20 which is the cost of the bus fare. However, the skilled person will understand that the cost of travel of a bus, or any other mode of public transport, may alternatively be calculated based on an average cost per unit distance.

A fourth weighting is also applied, wherein the fourth weighting is inversely proportional to the number of available substitute products for the navigation route. A fifth weighting is also applied, wherein the fifth weighting is inversely proportional to the product cost, wherein the product cost is calculated by the sum of the indications of the price of each available product navigated to by the navigation route. The skilled person will understand that these are mere examples of weighting types, and that other weightings based on alternative properties of the available products or navigation routes, or other combinations of these weightings, may also be used.

An example table illustrating the weightings applied to each of the example navigation routes from table 1 are included in table 2 below.

TABLE 2

| Nav. | Distance | Product | 1^{st} | 2^{nd} | 3^{rd} | 4^{th} | 5^{th} |
|---|---|---|---|---|---|---|---|
| route | (miles) | cost | weighting | weighting | weighting | weighting | weighting |
| 1 | 0.5 | [symbol] 7.08 | 0.90 | 1 | 1 | 1 | 0.29 |
| 2 | 0.5 | [symbol] 6.88 | 0.90 | 1 | 1 | 1 | 0.31 |
| 3 | 0.4 | [symbol] 6.88 | 0.92 | 1 | 1 | 0.33 | 0.31 |
| 4 | 3 | [symbol] 6.15 | 0.40 | 0.5 | 0.20 | 1 | 0.39 |
| 5 | 3.2 | [symbol] 6.49 | 0.36 | 0.3 | 0.3 | 1 | 0.36 |
| 6 | 3.5 | [symbol] 6.59 | 0.30 | 0.3 | 0.28 | 1 | 0.34 |

The processor 212 then identifies a recommended navigation route to obtain the plurality of products sought, based on the plurality of weightings. The remote server 210 then outputs the recommended navigation route (114) by sending a signal to the user equipment device 200 for communication to the user.

The skilled person will understand that the outputted recommended navigation route is be identified based on the plurality of weightings, however that the plurality of weightings need not be considered equally when identifying the recommended route. Indeed, each weighting type (e.g. first weighting, second weighting, etc.) may be subject to an additional significance weighting in order to identify the recommended navigation route. The weightings for identifying the recommended navigation route may be determined by user preferences, or by a set of pre-determined significance weightings. For example, the ranking of navigation routes may assign a higher weighting or significance to the fifth weighting, for example, to determine the recommended navigation route.

Purely for illustration, identifying the recommended navigation route may comprise applying the below significance weightings shown in Table 3 below to each weighting type. The skilled person will understand that these are mere examples of significance weightings, and that significance weightings may also be used.

TABLE 3

| Weighting type | Significance weighting |
|---|---|
| 1^{st} weighting (distance) | 0.2 |
| 2^{nd} weighting (mode of transport) | 0.1 |
| 3^{rd} weighting (cost of transport) | 0.15 |
| 4^{th} weighting (number of substitutes) | 0.3 |
| 5^{th} weighting (product cost) | 0.5 |

The recommended navigation route may then be identified of selected based on an aggregate score, wherein the aggregate score for each navigation route may be determined by the sum of the product of each weighting type and the corresponding significance weighting.

Purely as an example, the aggregate weightings calculated for each of the navigation routes discussed with reference to Tables 1 to 3 are shown in Table 4 below. Based on the scores shown in Table 4, navigation route 2 would be output as the recommended navigation route to the user device 200, wherein the user navigates to Shop A for milk, and Shop B for sourdough bread and digestive biscuits.

TABLE 4

| Navigation route | Aggregate score |
|---|---|
| 1 | 0.875 |
| 2 | 0.885 |
| 3 | 0.688 |
| 4 | 0.655 |
| 5 | 0.627 |
| 6 | 0.602 |

Whilst the method 100 described above is disclosed as being performed at the remote server 210, the skilled person will understand that in other embodiments the method 100 may be performed by the processor 202 of the user equipment device 200 instead. In such embodiments performed by the processor 202 of a user equipment device 200, the plurality of indications based on the indication of the plurality of products sought by the user may be retrieved by the processor 202 from the remote server 210, via the wireless communication means 204.

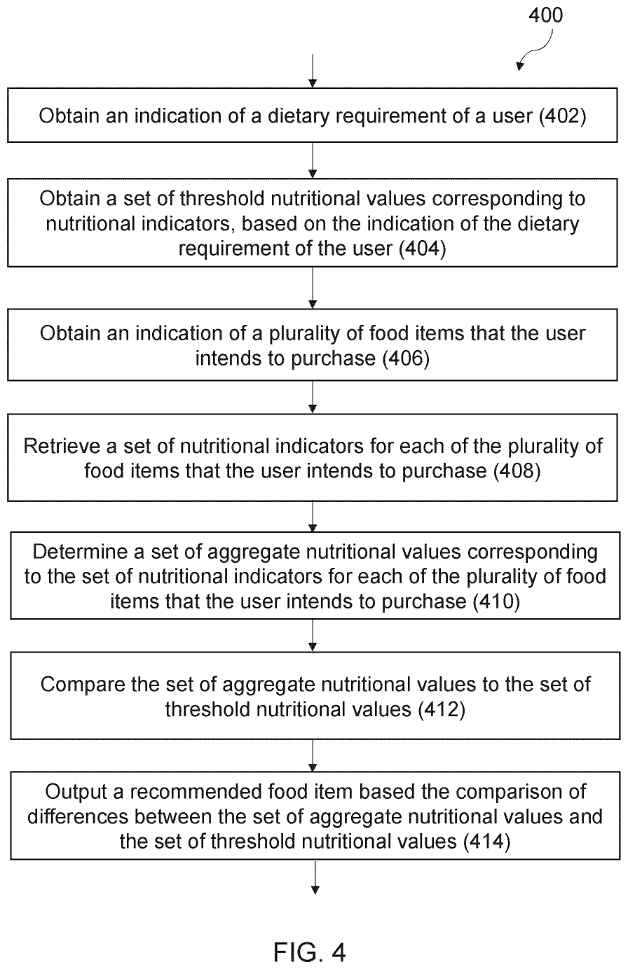

FIG. 4 shows a schematic flow diagram illustrating another example method 400 of the present invention. This is also described with reference to FIG. 2 which shows a schematic of an example system configured to perform the method 400.

In the present case, the remote server 210 obtains an indication of a dietary requirement of a user (402). This may be obtained via user input into the user equipment device 200, for example via a touch screen, keypad, or any other user input means which is then sent as a signal to the remote server 210 via the wireless communication means 204, or alternatively the indication may be retrieved from a memory 214 of the remote server 210 based on an indication of the identity of a user.

The processor 212 of the remote server 410 also obtains a set of threshold nutritional values corresponding to nutritional indicators, wherein the set of threshold nutritional values is based on the indication of the dietary requirement of the user (404). This may be obtained from the memory 414 of the remote server 410. The set of threshold nutritional values may correspond to nutritional guidelines based on the dietary requirement of the user, for example such as recommended daily or weekly nutritional targets. For example, the set of threshold nutritional values may comprise recommended values for a set of nutritional parameters, including but not limited to protein, carbohydrate, sugar, unsaturated fat, saturated fat, sodium, calcium, vitamin C, vitamin A, vitamin D, vitamin B12, fluid, or any other nutritional parameter.

Purely as an example, table 5 comprises sets of threshold nutritional values, for a variety of dietary requirements of a user. In this example, the dietary requirements of the user may be differentiated based on age and sex of the user, however the skilled person will understand that this is merely an example and that the dietary requirement of a user may, additionally or instead, be determined by other user characteristics, including but not limited to: dietary restrictions, such as vegan, vegetarian, etc.; illness or other health condition of the user, such as diabetic, obese, pregnant; food intolerances, such as coeliac, lactose-intolerant, and/or food allergies, such as allergies to wheat, soy, egg, milk, nuts, etc. This may be advantageous due to differing dietary requirements based on all of the above, age, sex, dietary restrictions, health conditions, and allergies.

TABLE 5

| | Threshold nutritional values is based on dietary requirement (years of age /sex) | | | | | | | | | | | | | | | |
|---|---|---|---|---|---|---|---|---|---|---|---|---|---|---|---|---|
| Nutritional | | | 12-18/ | | 12-18/ | | 18-65/ | | 18-65/ | | 65+/ | | 65+/ | | | |
| indicator | 4-12 | | Male | | Female | | Male | | Female | | Male | | Female | | Pregnant | |
| Protein | 19 | g | 52 | g | 46 | g | 55 | g | 45 | g | 90 | g | 60 | g | 46 | g |
| Carbs | 130 | g | 130 | g | 130 | g | 130 | g | 130 | g | 130 | g | 130 | g | 135 | g |
| Sugar | 19 | g | 25 | g | 25 | g | 30 | g | 30 | g | 30 | g | 30 | g | 30 | g |
| Unsaturated | 25 | g | 20 | g | 20 | g | 20 | g | 13 | g | <20 | g | <13 | g | 20 | g |
| Sodium | 1 | g | 2 | g | 2 | g | 2 | g | 2 | g | 2 | g | 2 | g | 3 | g |
| Calcium | 1 | g | 1.3 | g | 1.3 | g | 1 | g | 1 | g | 1.2 | g | 1.2 | g | 1-1.3 | g |
| Vitamin C | 15 | mg | 65 | mg | 65 | mg | 2 | g | 2 | g | 90 | mg | 75 | mg | 80 | mg |
| Vitamin A | 500 | mcg | 700 | mcg | 600 | mcg | 700 | mcg | 600 | mcg | 700 | mcg | 600 | mcg | 400 | mcg |
| Vitamin D | 10 | mcg | 10 | mcg | 10 | mcg | 10 | mcg | 10 | mcg | 10 | mcg | 10 | mcg | 10 | mcg |

The processor 212 then comprises obtaining an indication of a plurality of food items that the user intends to purchase (406). Again, this may be obtained via user input into the user equipment device 200, for example via a touch screen, keypad, or any other user input means which is then sent as a signal to the remote server 210 via the wireless communication means 204.

The processor 212 then retrieves a set of nutritional indicators for each of the plurality of food items that the user intends to purchase (408). The set of nutritional indicators may be retrieved, for example, from the memory 214 of the remote server, or by mining information from the internet relating to each of the food items sought.

Based on the set of nutritional indicators for each of the plurality of food items that the user intends to purchase, the processor 212 determines a set of aggregate nutritional values corresponding to the set of nutritional indicators for each of the plurality of food items that the user intends to purchase (410).

The processor 212 compares the set of aggregate nutritional values to the set of threshold nutritional values and identifies differences between the set of aggregate nutritional values and the set of threshold nutritional values (412).

In the event that at least one of the aggregate nutritional values from the set of aggregate nutritional values is different to the corresponding threshold nutritional value, the processor 212 identifies at least one recommended food item, wherein the at least one recommended food item is identified based on a set of nutritional indicators for the at least one recommended food item which correspond to at least one of the differences between the set of aggregate nutritional values for the food items intended to buy and the set of threshold nutritional values.

This may be advantageous to output recommended food items to ensure a user's recommended nutritional requirements are being met based on their dietary requirements, thereby helping to reduce nutritional deficiencies of the user.

The identified recommended food item is then output by the remote server 210 to the user equipment device 200 for display to the user (414).

Preferably, the outputted at least one recommended food item is a substitute food item corresponding to at least one of the plurality of food items that the user intends to purchase. However, the skilled person will understand that the outputted at least one recommended food item may alternatively be an additional food item to supplement the plurality of food items that the user intends to purchase. For example, if the indication of the dietary requirements of the user include an indication that the user is vegan, and the indication of the plurality of products sought by the user comprises an oat drink, the method 400 may output a recommended substitute product of an oat drink fortified with vitamin B12, due to the common B12 deficiency among vegans and vegetarians.

Whilst the method described above is disclosed as being performed at the remote server 210, the skilled person will understand that in other embodiments the method 100 may be performed by the processor 202 of the user equipment device 200 instead.

In addition, whilst the methods 100 and 400 are described above separately, the skilled person will understand that these methods can, in some embodiments of the present invention, be combined.

For example, in one embodiment, the processor 212 obtains an indication of the geographical location of the user equipment device 200, and thereby the geographical location of the user (102), an indication of a dietary requirement of a user (402), and a set of threshold nutritional values corresponding to nutritional indicators, wherein the set of threshold nutritional values is based on the indication of the dietary requirement of the user (404).

The processor 212 also obtains an indication of a plurality of food items sought by the user (104, 406). This may be obtained via user input into the user equipment device 200, for example via a touch screen, keypad, or any other user input means. This obtained user input may then be sent as a signal to the remote server 210 via the wireless communication means 204.

The processor 212 then retrieves a set of nutritional indicators for each of the plurality of food items that the user intends to purchase (408). Based on the set of nutritional indicators for each of the plurality of food items that the user intends to purchase, the processor 212 determines a set of aggregate nutritional values corresponding to the set of nutritional indicators for each of the plurality of food items that the user intends to purchase (410).

The processor 212 compares the set of aggregate nutritional values to the set of threshold nutritional values and identifies differences between the set of aggregate nutritional values and the set of threshold nutritional values (412). The processor 212 then identifies and outputs at least one recommended substitute food item, wherein the at least one recommended substitute food item is identified based on a set of nutritional indicators for the recommended substitute food item which correspond to at least one of the differences between the set of aggregate nutritional values for the food items intended to buy and the set of threshold nutritional values (414).

The processor 212 then updates the indication of the plurality of food items sought by the user, based on the recommended substitute food items. Thus, the updated indication of the plurality of food items sought by the user comprises the recommended substitute food items.

This may be advantageous to output recommended substitute food items to improve the nutritional value of food sought by a user and to ensure the user's recommended nutritional requirements are being met based on their dietary requirements, thereby helping to reduce nutritional deficiencies of the user.

Based on the updated indication of a plurality of food items sought by the user, the processor 212 is configured to retrieve a plurality of indications, wherein each of the plurality of indications relates to an available product corresponding to one of the plurality of food items sought in the updated indication (106). Each indication of an available product also comprises an indication of the geographical location of said available product, and an indication of the price of the available product.

The processor 212 groups said plurality of indications into a plurality of destinations, based on the geographical location of each available product. Each destination may correspond, for example, to a store comprising at least one available product (108).

The processor 212 then determines a plurality of navigation routes, each navigation route is configured to navigate from the geographical location of the user to at least one of the destinations (110), such that each navigation route is configured to navigate to a plurality of available products corresponding to each of the plurality of products sought by the user.

The processor 212 then applies a plurality of weightings to each navigation route, and outputs at least one recommended navigation route, or plurality of destinations within a navigation route, based on the plurality of weightings. The skilled person will understand that the weightings may be applied as described with reference to FIG. 1, or any other suitable permutation thereof.

For example, a first weighting is applied wherein the first weighting is inversely proportional to the distance of the navigation route (112). A second weighting is also applied, wherein the second weighting is inversely proportional to the product cost, wherein the product cost is calculated by the sum of the indications of the price of each available product navigated to by the navigation route. In addition, a third weighting may be applied based on the number of available recommended substitute food items. This may be advantageous to favourably weight navigation routes comprising the outputted recommended substitute products based on the user's dietary requirements. Alternatively, a weighting may be applied based on nutritional values of each indication of an available product within a navigation route, weighted relative to the set of threshold nutritional values. This may be advantageous to favourably weight navigation routes and products based on their nutritional value and the user's specific dietary requirements. This may be advantageous to improve the nutritional value of food sought by a user and to ensure the user's recommended nutritional requirements are being met based on their dietary requirements, thereby helping to reduce nutritional deficiencies of the user.

The skilled person will understand that these are mere examples of weighting types, and that other weightings based on alternative properties of the available products or navigation routes, or other combinations of these weightings, may also be used.

The processor 212 then identifies a recommended navigation route to obtain the plurality of products sought, based on the plurality of weightings. This recommendation is then output by the remote server 210 (114) and sent to the user equipment device 200 for communication to the user.

As described with reference to FIG. 1, the skilled person will understand that the outputted recommended navigation route is be identified based on the plurality of weightings, however that the plurality of weightings need not be considered equally when identifying the recommended route. Indeed, each weighting type (e.g. first weighting, second weighting, etc.) may be subject to an additional significance weighting in order to identify the recommended navigation route.

Whilst the method described above is disclosed as being performed at the remote server 210, the skilled person will understand that in other embodiments the method 100 may be performed by the processor 202 of the user equipment device 200 instead.

Embodiments of the present invention may therefore increase efficiency of obtaining desired products, reduce food waste, and improve user health based on providing recommended food substitutes based on the dietary requirements of the user. Embodiments of the present invention may also reduce last mile carbon emissions for a user to obtain desired products, whilst also optimising consumer time and cost efficiencies to obtain said desired products.

In the context of the present disclosure other examples and variations of the apparatus and methods described herein will be apparent to a person of skill in the art.

1. A computer-implemented method for determining a navigation route configured to obtain a plurality of products sought by a user, the method comprising:
obtaining an indication of a geographical location of a user;
obtaining an indication of a plurality of products sought by the user;
retrieving a plurality of indications based on the indication of the plurality of products sought by the user, wherein each of the plurality of indications relates to an available product, and wherein each indication of an available product comprises an indication of
the geographical location of said available product;
grouping the plurality of indications each relating to an available product into a plurality of destinations based on the geographical location of the available products; determining a plurality of navigation routes from the geographical location of the user, wherein each navigation route is configured to navigate to at least one destination of the
plurality of destinations, such that each complete navigation route is configured to navigate
to a plurality of available products corresponding to each of the plurality of products sought
by the user;
applying a first weighting to each of the plurality of navigation routes, wherein the first weighting is inversely proportional to at least one of (i) time and (ii) distance of the
navigation route; and
outputting at least one recommended navigation route configured to obtain the plurality of products sought by the user, based on the first weighting applied to each of the plurality of navigation routes.

2. The method of claim 1 wherein determining the plurality of navigation routes comprises determining a plurality of navigation routes from the geographical location of the user for a plurality of modes of transport, the method further comprising:
applying a second weighting to each of the plurality of navigation routes, wherein the second weighting is based on the mode of transport of the route; and
wherein outputting the at least one recommended navigation route is based on the
first weighting and the second weighting applied to each of the plurality of navigation routes.

3. The method of claim 1 wherein each indication of the plurality of indications relating to an available product relates to one of: (i) an available product corresponding to one of the plurality of products sought by the user; and (ii) an available substitute product corresponding to one of the plurality of products sought by the user:
the method further comprising:
for each of the plurality of navigation routes, counting the number of available substitute products for the navigation route:
applying a third weighting to each of the plurality of navigation routes, wherein the third weighting is inversely proportional to the number of available substitute products for
the navigation route; and
wherein outputting the at least one recommended navigation route is additionally based on the third weighting applied to each of the plurality of navigation routes.

4. The method of claim 1 further comprising:
identifying at least one destination which does not comprise an indication relating to an available product for all of the plurality of products sought by the user;
for said destination, determining at least one missing product of the plurality of products sought by the user, wherein said destination does not comprise an indication relating to an available product for the at least one missing product;
retrieving, for said destination, a second plurality of indications each relating to an available substitute product, wherein the second plurality of indications are retrieved based on the at least one missing product;
determining a second plurality of navigation routes from the geographical location of the user, wherein each navigation route of the second plurality of navigation routes is
configured to navigate to at least one destination of the plurality of destinations, such that
each complete navigation route is configured to navigate to (i) at least one available product, and (ii) at least one available substitute product, such that the at least one available product and the at least one available substitute product correspond to the plurality of products sought by the user;
applying a first weighting to each of the second plurality of navigation routes, wherein the weighting is inversely proportional to at least one of (i) time and (ii) distance of the navigation route;
applying a second weighting to each of the second plurality of navigation routes,
wherein the weighting is inversely proportional to the number of available substitute products for the navigation route; and
wherein outputting the at least one recommended navigation route is based on the weighting applied to each of the plurality of navigation routes, including the second plurality
of navigation routes.

5. The method of claim 1, wherein each indication of an available product further comprises an indication of at least one of property of said product: the method further comprising:
obtaining a set of user preferences, wherein the set of user preferences correspond to preferred properties of at least a portion of the plurality of products sought by the user: determining a first weighting for each indication of an available product, based on the at least one property of the available product and the set of user preferences;
for each of the plurality of navigation routes, determining fourth weighting, wherein
the fourth weighting is based on the first weighting of each indication of an available product navigated to by the navigation route; and
wherein outputting the at least one recommended navigation route is additionally based on the fourth weighting applied to each of the plurality of navigation routes.

6. A computer-implemented method for outputting an indication of at least one recommended available product from a plurality of available products from disparate sources, the method comprising:
obtaining an indication of a geographical location of a user; obtaining an indication of a product sought by a user;
retrieving a plurality of indications each relating to an available product, wherein the
plurality of indications are retrieved based on the indication of the product sought by the user, wherein each indication of an available product comprises an indication of the product price, and an indication of the geographical location of the product;
applying a first weighting to each of the plurality of indications relating to an
available product, based on the indication of the product price;
for each of the plurality of indications relating to an available product, determining at least one navigation route from the geographical location of the user to the geographical location of the available product;
applying a second weighting to each navigation route, wherein the second weighting is inversely proportional to at least one of (i) time and (ii) distance of the navigation route to the geographical location of the available product;
outputting an indication of at least one recommended available product, based on
the first weighting and the second weighting.

7. The method of claim 6 wherein determining at least one navigation route from the geographical location of the user to the geographical location of the product comprises determining a plurality of navigation routes for a plurality of modes of transport; and
wherein the second weighting applied to each navigation route comprises: a first component being inversely proportional to at least one of (i) time and (ii) distance of the navigation route to the geographical location of the available product, and a second component based on the mode of transport of the route.

8. (canceled)

9. (canceled)

10. (canceled)

11. (canceled)

12. A computer readable non-transitory storage medium comprising a program for a computer configured to cause a processor to perform the method of claim 1.

13. An inventory monitoring device, comprising:
a sensor configured to be mounted to a shelf, wherein the sensor is configured to obtain an indication of the weight of product stock stored on the shelf;
a processor configured to detect when at least one product from the product stock is removed from the shelf based on the indication of the weight of product stock on the shelf obtained by the sensor; and
a communication means configured to send a signal to a remote device, wherein the signal includes the indication of the at least one product which has been removed from the product stock on the shelf.

14. A computer readable non-transitory storage medium comprising a program for a computer configured to cause a processor to perform the method of claim 6.
